(19) United States
(12) Patent Application Publication
(10) Pub. No.: US 2022/0379070 A1
(43) Pub. Date: Dec. 1, 2022

(54) ADJUSTABLE VALVE ASSEMBLY FOR MEDICAL TUBING

(71) Applicant: Orlando V. Morejon, Palmetto Bay, FL (US)

(72) Inventor: Orlando V. Morejon, Palmetto Bay, FL (US)

(21) Appl. No.: 17/828,906

(22) Filed: May 31, 2022

Related U.S. Application Data
(60) Provisional application No. 63/194,664, filed on May 28, 2021.

(51) Int. Cl. A61M 16/20; A61M 16/04

(52) U.S. Cl. CPC A61M 16/208; A61M 16/0465

(57) ABSTRACT

A valve assembly for adjustably regulating communication with medical tubing applied to a patient including a housing having a hollow interior and a primary connector interconnecting the medical tubing to the hollow interior. First and second ports are attached to and movable with a closure segment, concurrently and alternately, between an open orientation and a closed orientation. The concurrent and alternate disposition of the first and second ports comprising each disposed in the open orientation concurrent to the other disposed in the closed orientation. The open orientation of each of said first and second ports comprising disposition thereof in communicating relation with the medical tubing, via said hollow interior and said primary connector.

### BACKGROUND OF THE INVENTION

The present application is based on, and a claim of priority is made under 35 U.S.C. Section 119(e) to a provisional patent application that is currently pending in the U.S. Patent and Trademark Office, namely, that having Ser. No. 63/194,664 and a filing date of May 28, 2021, and which is incorporated herein by reference.

### FIELD OF THE INVENTION

A valve assembly structured to adjustably regulate access to and communication with medical tubing including, but not limited to, endotracheal tubing operatively applied to a patient.

### DESCRIPTION OF THE RELATED ART

Many patients in a hospital, including patients in an Intensive Care Unit (“ICU”), must be fitted with an artificial airway, such as an endotracheal tube, to facilitate their respiration. Typically, the artificial airway tube comprises an elongate, semi-rigid lumen which is inserted into a patient's nose or throat and projects down into airflow communication with the patient's respiratory system. As such, the patient either directly, or with the aid of a respiratory unit, is able to breathe more effectively through the artificial airway tube. Also, a manifold or connector structure is used to connect the artificial airway or endotracheal tube to a source of breathable gas.

Recent studies have determined, however, that the accumulation of dried tracheo-bronchial secretions on the interior wall surface of an operating artificial airway tube effectively decreases the lumen cross section, and thereby significantly increases the work of breathing for the intubated patient. Moreover, increasing the work of breathing for the patient necessitates that a higher level of support be provided to compensate, and often results in the patient's intubation period and ICU stay being significantly prolonged. Furthermore, it is also seen that thick secretions on the walls of the artificial airway tube often serve as a nidus for continued infection in the lungs, leading to added morbidity and hospital costs for the intubated patient.

Therefore, while a patient is attached to the respiratory support system, it is periodically necessary to aspirate fluids and/or secretions from the patient's trachea and lungs. In the performance of prior art techniques, it was frequently necessary to disassemble part of the respiratory support system by removing the ventilator manifold/connector and inserting an intervention device such as, but not limited to, a suction catheter into the artificial airway. Fluid and like contaminants were then suctioned from the patient. Thereafter, the suction catheter was removed and the respiratory support system, including the manifold/connector, was reassembled. However, due to the interruption of respiratory support during such an interventional procedure, a patient's blood oxygen can often drop and carbon monoxide can reach an unacceptable level. Additionally, unless a sufficient positive end expiratory pressure (PEEP) level is maintained there is a possibility that the lungs of the patient will collapse. Moreover, procedures of this type may have to be performed numerous times per day and during relatively long periods of hospitalization of the patient.

In order to overcome well recognized problems of the type set forth above it is important to provide respiratory equipment that will minimize patient discomfort. In addition, improvement in the equipment and/or operative components of a respiratory and or ventilation system should have sufficient operative versatility to treat patients of different age groups. In recognizing such procedural problems including the maintaining adequate pressure in a ventilation system, numerous attempts have been made to provide improved facilities which serve to maintain a continuous flow of oxygen from the respirator device through aforementioned manifold/connector and the artificial airway tube to the lungs of the patient. Such known attempts intended to be operative to facilitate the insertion and retraction of various types of intervention devices such as, but not limited to, the aforementioned suction catheter. However, known attempts of this type failed to provide an operable system capable of performing machine assisted respiration without disconnecting the respirator a disassembly of the interposed manifold/connector.

Accordingly, there is a need in the medical profession for a connector assembly structured for use with a medical ventilator system, wherein a completely closed ventilator circuit is established and maintained from the start of mechanical ventilation until the patient is ready for removal from ventilator support and/or removal of the artificial airway. Moreover, an improved and proposed connector assembly should be structured to include a plurality of ports including, but not limited to, an intervention port. As such, the introduction of any one or more of a variety of different intervention devices will be facilitated in a manner which avoids the loss of airway pressure (PEEP) during all types of interventional procedures involving the airway and transitional steps associated therewith. Moreover, the structural and operative features of such a proposed connector assembly and associated ventilation components eliminates interruptions in the patient's ventilator support and oxygenation while preserving adequate pressure during all interventional procedures.

### SUMMARY OF THE INVENTION

The present invention is directed to a valve assembly structured for adjustably regulating communication with medical tubing including, but not limited to, endotracheal tubing when applied to a patient.

The valve assembly includes a housing having a hollow interior and a primary connector. The primary connector is disposed on and/or connected to the housing and structured for attachment of the housing to the aforementioned medical and/or endotracheal tubing as such, the primary connector establishes fluid communication with the hollow interior of the housing and the interior of the endotracheal tubing. In addition, a first port and a second port are movably connected to the housing and are concurrently and alternatively disposed in an open orientation and a closed orientation.

In more specific terms, the “concurrent and alternate” disposition of the first and second ports comprises and is defined herein as each of the first and second ports being disposed in the open orientation concurrent to the other of the first and second ports disposed in the closed orientation. As a result, only one of the first and second ports is disposed in communicating relation with the interior of the housing at a time, while the other of the first and second ports are disposed in the closed orientation. As should be apparent and as described in greater detail hereinafter disposition of either the first and second ports in the open orientation establishes a communicating relation with the medical tubing via the hollow interior of the housing and the primary connector.

In terms of the first and second port being movably and adjustably disposed, concurrently and alternatively in the open and closed orientations, such movement or adjustability is accomplished by and at least minimal rotation of the first and second ports substantially about an exterior of the housing and the hollow interior thereof. Accordingly, each of the one or more preferred embodiments of the valve assembly of the present invention includes a port supporting segment or “closure segment” movably and more specifically rotationally connected to the housing. Moreover, each of the first and second ports are fixedly and or integrally connected to the closure segment so as to be concurrently movable therewith into the aforementioned concurrent and alternative disposition between open and closed orientations.

The housing of the valve assembly also includes an access opening formed therein in direct access and/or communication with the hollow interior of the housing. In cooperation there with, the closure segment is dimensioned, configured and structured to be disposed within and/or in covering, enclosing relation to the access opening as it is rotationally adjustable relative thereto. Therefore, regardless of either the first or second port being disposed in the open orientation, the closure segment will be in closing, covering and at least partially sealing relation to the access port and in at least partially enclosing relation to the hollow interior. Moreover, the cooperative structural features between the closure segment and the access opening are sufficient to establish at least a minimal sealing relation therebetween. Such a sealing relation or engagement, while not necessarily being specifically air-tight, is sufficient to facilitate uninterrupted fluid flow through the hollow interior of the housing, to and from the patient via the applied medical or endotracheal tubing.

Each of one or more of the operative embodiments of the valve assembly of the present invention include a closure assembly disposed and structured to established and defined the closed orientation of the first and second ports. The closure assembly comprises a first closure member fixedly connected to the housing in at least partially covering relation to the access opening and further disposed in closing relation with at least one of the first and second ports upon predetermined rotational movement of the closure segment and the first and second ports connected thereto within the access opening. Such closing relation between the first closure member and a corresponding one of the first and second ports comprises the open inner end of the corresponding port being disposed in overlying and/or covering relation to the first closure member. As emphasized herein the first closure member being disposed in the closing relation to one of the first and second ports will occur concurrently to the other of the first and second ports being disposed in the open orientation in communication with the hollow interior the housing and the medical tubing, as set forth herein.

The closure assembly further comprises a second closure member, which may be defined by a sidewall segment or portion of the housing disposed immediately adjacent to and defining a correspondingly disposed border or periphery of the aforementioned access opening. Similar in operation to the first closure member, closing relation of the second closure member with a different one of the first and second ports comprises the open inner end of the corresponding other port being disposed in overlying, covering relation to the second closure member. As set forth above, the second closure member being disposed in closing relation to one of the first and second ports will occur concurrently to the other of the first and second ports being disposed in the open orientation, in communicating relation with a hollow interior of the housing and the medical tubing.

With further regard to the first and second port, each may include an external attachment segment extending outwardly from the housing. Each such attachment segment may be dimensioned and configured to establish appropriate connection and attachment different sources/devices which facilitate introduction of fluid or instrumentation into the interior of the medical tubing via passage through the hollow interior of the housing and the primary connector. Accordingly, in more specific terms one of the first and second ports is structured to define a fluid flow port into the interior of the housing and in communication with the medical tubing via the primary connector. In cooperation there with, the other of the first and second ports may be structured to define an intervention port for the introduction of instrumentation and/or other medical devices into the interior of the medical tubing via the hollow interior of the housing and the primary connector.

As is recognized, such instrumentation or medical devices may be used to facilitate cleaning, maintenance, etc. of the medical tubing concurrent to its application to the patient. The other the first and second ports operative as a fluid flow port may also be connected to a vacuum or source of negative pressure thereby facilitating removal of any substances from the interior of the medical tubing, concurrent to its being applied to the patient.

Additional structural and operative features of the valve assembly include one or more embodiments thereof including a cover assembly attached to or integrated in the structure of the housing such that the opposite longitudinal ends of the housing are covered or enclosed and thereby segregate the hollow interior of the housing from an exterior thereof.

However, one or more additional embodiments of the adjustable valve assembly of the present invention comprises the valve assembly and more specifically the housing having a T-shape configuration. Moreover, the T-shape housing includes at least one but more practically a plurality of at least two supplementary ports each disposed in fluid communication with the interior of the housing and with the interior of the medical tubing via the primary connector. Further, each of the preferred two supplementary ports are disposed at opposite longitudinal ends of the housing and may be disposed, structured and configured for operative connection with breathing apparatus facilitating the inflow and outflow of fluid, as the patient is breathing. Accordingly, the T-shape embodiment of the adjustable valve assembly facilitates maintenance thereof in operative attachment to the patient while selective disposition of the first and second port in communication with the medical tubing via the hollow interior of the housing and the primary connector.

Therefore, the structural and operative features of the adjustable valve assembly of the present invention and eliminate the need to disassemble part of the respiratory support system for purposes of adding or connecting equipment intended maintain the medical tubing and its operation. Such intervention, as indicated above may include, but not limited to, a suction catheter into the artificial airway. Fluid and like contaminants can then be suctioned from the patient without disconnection of the respiratory support system and eliminate dangerous conditions. It has been commonly recognized that the interruption of respiratory support during such an interventional procedure may result in a patient's blood oxygen often dropping and carbon monoxide possibly reaching unacceptable levels. Further, unless a sufficient positive and expiratory pressure (PEEP) level is maintained there is a possibility that the lungs of the patient will collapse. Moreover, procedures of this type may have to be performed numerous times per day and during relatively long periods of hospitalization of the patient.

Yet additional features of the assembly of the present invention includes structure associated with maintaining sterility thereof and/or instrumentation passing through the valve assembly into the interior of endotracheal or other medical tubing disposed within the interior of the patient, such as in the bronchial area. More specifically, one or more embodiments of the present invention may include an elongated flexible, collapsible material sleeve connected to at least one or both of the first and second ports associated with the valve assembly, as set forth above. In cooperation there with an opposite end of the sleeve is connected to an at least partially movable with the suction catheter or other instrument passing through the first or second port into the interior of the body of the adjustable valve assembly. The sleeve and elongated configuration is sufficient to enclose at least a majority of the length of the catheter assembly and a portion thereof which extends into the medical tubing, via the valve assembly. Moreover, prior to use or application of a suction catheter assembly or other instrumentation, the sleeve is connected at one of its ends to a portion of the catheter assembly adjacent to a proximal end of a portion of the instrumentation passing into the interior of the medical tubing within the patient. In its initial orientation, prior to use, the length of the sleeve is sufficient to cover and/or enclose an entirety or at least a majority of the length of the inserted portion of the instrumentation. In at least one embodiment the sleeve extends over and in the enclosing relation to the inserted length. As a result, the sterility of the catheter assembly and in particular the portion thereof that is inserted through the valve assembly and into the lumen of the medical tubing will be maintained in sterile condition, prior to use.

In order to maintain its intended operative features, the opposite or distal end of the sleeve is secured to the corresponding first or second port through which the suction catheter assembly or other instrumentation passes as it enters the interior of the medical tubing within the patient. As described in greater detail hereinafter, the adjustable valve assembly having the flexible material sleeve attached thereto may or may not be attached to and accompany the suction catheter assembly upon removal from packaging and prior to use.

Therefore, a preferred fixed connections of opposite ends of the sleeve to the suction catheter assembly and to the adjustable valve assembly, as well as the flexible material from which the sleeve is formed, facilitates the inward collapse, compacting and/or folding of the sleeve upon itself, concurrently to the intended instrumentation being introduced through the first or second port of the adjustable valve assembly and into the interior of the endotracheal tube or other medical tubing within the patient. As indicated above, the inherent flexibility of the material from which the elongated sleeve is formed facilitates its “inward” collapse or folding of the sleeve upon itself at the same time the instrumentation is passing into and through the adjustable valve assembly. After the suction catheter assembly has performed the intended drainage/cleaning procedure from the interior of the endotracheal tube or other medical tubing within the patient, the withdrawing of the catheter assembly medical tubing, through the valve assembly, will result in the concurrent expansion and/or elongation of the sleeve into its original elongated configuration. Such elongation of the sleeve will dispose it in the original overlying, enclosing relation to the inserted portion of the catheter assembly upon the respective withdrawal thereof from the medical tubing and the adjustable valve assembly. As a result, the sleeve will then be disposed in overlying covering and somewhat isolating relation to the inserted portion of the catheter assembly thereby allowing it to be reused, while maintaining the sterility thereof, except for exposure to the body lumen or medical tubing. These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a front perspective view of one embodiment of the valve assembly of the present invention.

FIG. 2 is a perspective view in exploded form of the embodiment of FIG. 1.

FIG. 3 is a top perspective view of the embodiment of FIGS. 1-2.

FIG. 4 is a bottom perspective view of the embodiment of FIGS. 1-3

FIG. 5 is a rear side perspective view of the embodiment of FIGS. 1-4.

FIG. 6 is a front perspective view of another embodiment of the valve assembly of the present invention.

FIG. 7 is a rear perspective view of the embodiment of FIG. 6.

FIG. 8 is a top perspective view of the embodiment of FIGS. 6 and 7.

FIG. 9 is a perspective view in exploded form of the embodiment of FIGS. 6-8.

FIG. 10 is a perspective view of an additional structural modification of the embodiment of FIG. 1.

FIG. 11 is a perspective view of an additional structural modification of the embodiment of FIG. 7.

FIG. 12 is a perspective view of an additional preferred embodiment of the valve assembly of the present invention in an open orientation.

FIG. 13 is a perspective view of the embodiment of FIG. 12 in a closed orientation.

FIG. 14 is a perspective interior view of the embodiment of FIGS. 12 and 13.

FIG. 15 is a plan view in partial cutaway representing structural modifications of the embodiment of FIGS. 12-14.

Like reference numerals refer to like parts throughout the several views of the drawings.

### DETAILED DESCRIPTION OF THE PREFERRED EMBODIMENT

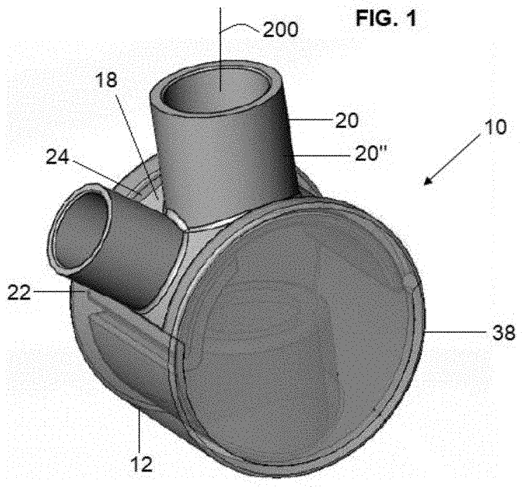

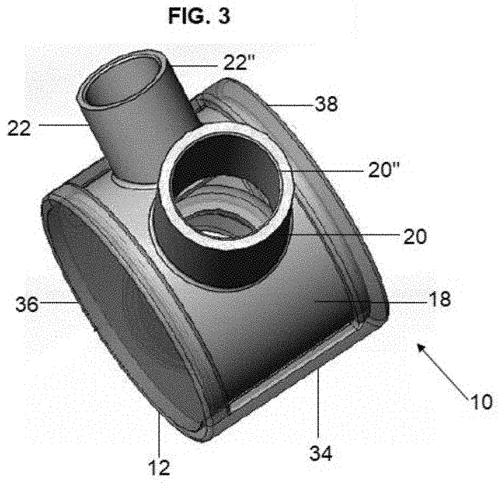

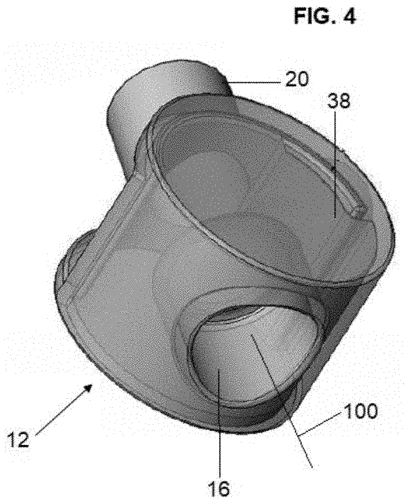

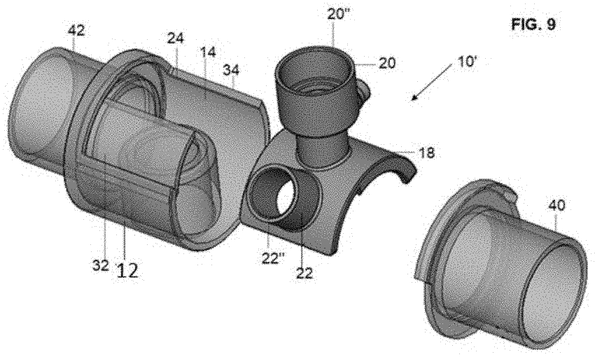

The present invention is directed to an adjustable valve assembly structured for regulating communication with medical tubing including, but not limited to, endotracheal tubing when operatively applied to a patient. More specifically, the valve assembly is generally represented as 10 and 10′ respectively in the embodiments of FIGS. 1-5 and 6-9. As such, the different embodiments of the valve assembly 10 and 10′ facilitate, but are not limited to, the operative introduction and application of an interventional procedure to the patient, without interruption or disconnection of respiratory support. Further, the versatility of the valve assembly 10 facilitates the introduction of the interventional procedure independent of respiratory support.

Accordingly, with primary reference to FIGS. 1-5, valve assembly 10 includes a housing 12 having a hollow interior 14 and a primary connector 16. The primary connector 16 is disposed on and/or connected to the housing 12 and structured for attachment of the housing to the aforementioned medical and/or endotracheal tubing schematically represented as 100. As such, the primary connector 16 establishes fluid communication with the hollow interior 14 of the housing 12 and the interior of the endotracheal tubing 100. In order to facilitate the establishment of effective communication, as described in greater detail herein, the primary connector 16 may extend into the hollow interior 14 in order to define alignment with either one of a first port 20 or a second port 22, which are incorporated in the embodiments of FIGS. 1-5 and 6-9, when disposed in an open orientation.

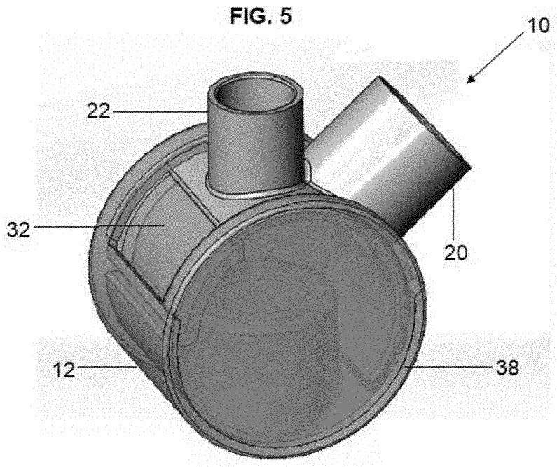

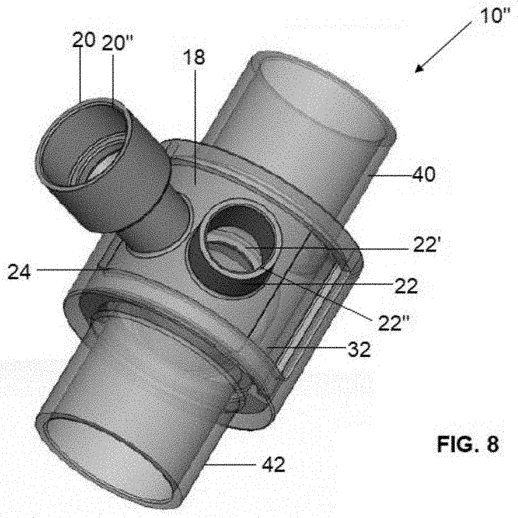

More specifically, the first port 20 and the second port 22 are movably connected to the housings 12 and 12′ and are alternatively disposed in an open orientation and a closed orientation. In more specific terms, the “concurrent and alternate” disposition of the first and second ports 20 and 22 comprises and is at least partially defined herein as each of the first and second ports 20 and 22 being disposed in an open orientation concurrent to the other of the first and second ports 20 and 22 disposed in the closed orientation. As represented in FIGS. 1 and 3-5, and FIGS. 6-8, the first port 20 is disposed in the open orientation concurrent to the second port 22 being disposed in the closed orientation. In contrast, FIGS. 5 and 8 represent the second port 22 being disposed in the open orientation concurrent to the first port 20 being disposed in the closed orientation.

When in the open orientation, each of the first or second ports 20 or 22 is disposed in direct communicating relation with the interior 14 of the housing 12 or 12′ and the interior of the medical or endotracheal tubing attached to the primary connector 16. As indicated above, the internal positioning of the primary connector 16 within the hollow interior 14 of the housing 12 and 12′ facilitates a substantially direct, un-interrupted communication between each of the first and second ports 20 and 22, when that first or second port 20 and 22 is disposed in the open orientation. As a result, only one of the first and second ports 20 and 22 is disposed in communicating relation with the interior 14 of the housing 12 or 12′ at a time, while the other of the first and second ports 20 and 22 are disposed in the closed orientation. As should be apparent and as set forth herein, disposition of either the first and second ports 20 and 22 in the open orientation establishes a communicating relation with the medical tubing via the hollow interior 14 of the housing 12 or 12′ and the primary connector 16.

In terms of the first and second port 20 and 22 being movably and adjustably disposed, “concurrently and alternately” in the open and closed orientations, such movement or adjustability is accomplished by an at least minimal rotation of the first and second ports 20, 22 substantially about an exterior of the housing 12 and the hollow interior 14. Accordingly, each of the one or more preferred embodiments of the valve assembly 10 and 10′ of the present invention includes a port supporting segment or “closure segment” 18 movably and more specifically rotationally connected to the housing 12 and 12′. Moreover, each of the first and second ports 20, 22 are fixedly and or integrally connected to the closure segment 18 so as to be concurrently movable/rotational therewith into the aforementioned concurrent and alternative disposition between open and closed orientations.

The housing 12 and 12′ of the valve assembly 10 also includes an access opening 24 formed therein in direct access and/or communication with the hollow interior 14 of the housing 12 and 12′. In cooperation therewith, the closure segment 18 is dimensioned, configured and structured to be disposed within and/or in covering, closing relation to the access opening 24, as it is rotationally adjusted relative to the housing 12 and its hollow interior 14. Therefore, regardless of either the first or second port 20, 22 being disposed in the open orientation, the closure segment 18 will be disposed in closing, covering and at least partially sealing relation to the access port 24 and in at least partially enclosing relation to the hollow interior 14. Moreover, the cooperative structural features between the closure segment 18 and the access opening 24 are sufficient to establish at least a minimal sealing relation therebetween. Such a sealing relation or engagement is sufficient to facilitate uninterrupted fluid flow through the hollow interior 14 of the housing 12 and 12′, to and from the patient via the applied medical or endotracheal tubing 100.

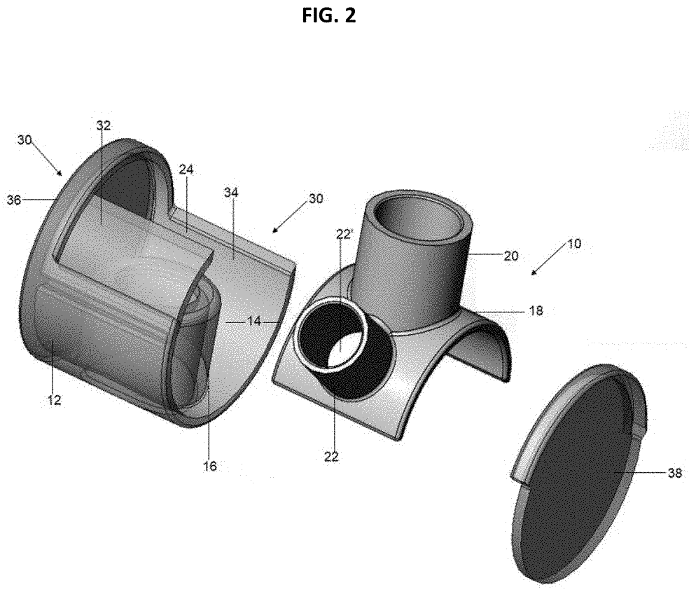

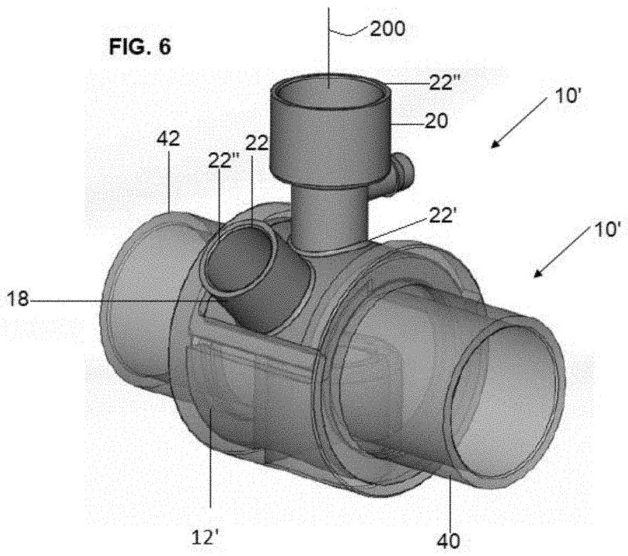

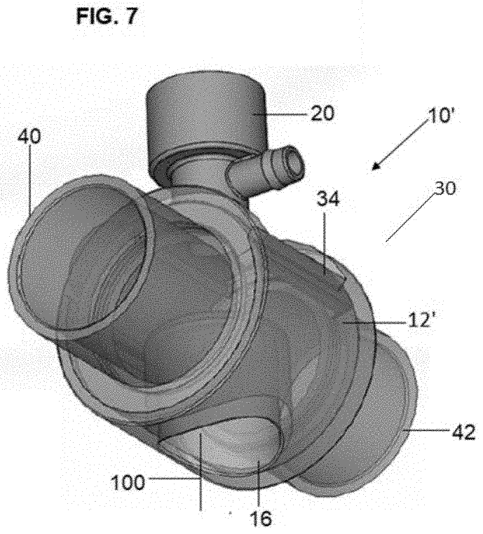

Two different operative embodiments of the valve assembly 10 and 10′ are respectively represented in FIGS. 2 and 7, wherein each include a closure assembly generally indicated as 30. The closure assembly 30 disposed and structured to establish and define the closed orientation of the first and second ports 20, 22. As represented in at least FIGS. 2, 5 and 7-9, the closure assembly 30 comprises a first closure member 32 fixedly connected to the housing 12 in at least partially covering relation to the access opening 24. The first closure member 32 is further disposed in closing relation with at least one of the first and second ports 20, 22 upon predetermined rotational movement of the closure segment 18 and the first and second ports 20, 22 connected thereto within the access opening 24. With reference to FIGS. 1 and 3-5, of the valve assembly 10 and FIGS. 6-9 of the valve assembly 10′, the first port 20 is disposed in the open orientation in communicating relation with the hollow interior 14 and the endotracheal tube 100 attached to the primary connector 16. Concurrently, the second port 22 is disposed in the closed orientation, wherein the first closure member 32 is disposed in covering, closing relation to the inner most open end 22′ of the second port 22. As emphasized herein, when the first closure member 32 is disposed in the closing relation to open inner end 22′ of the second port 22, the open inner end of the 20′ of the first port 20 will be concurrently disposed in the open orientation and direct in communication with the hollow interior 14 the housing 12 or 12′ and the endotracheal tubing, via the primary connector 16.

The closure assembly 30 further comprises the second closure member 34, which may be defined by a sidewall segment or portion of the housing 12 disposed immediately adjacent to and defining a correspondingly disposed border or periphery of the access opening 24. Similar in operation to the first closure member 32, the closed orientation of the first port 20 comprises the second closure member 34 being disposed in closing, covering relation to the open inner end 20′ of the first port 20. As set forth above, the second closure member 34 being disposed in closing relation to inner end 20′ of the first port 20, will occur concurrently to the second port 22 being disposed in the open orientation (see Figures Sand 8), in communicating relation with a hollow interior 14 of the housing 12 or 12′ and the medical tubing, via the primary connector 16.

Therefore the “concurrent and alternate disposition” comprising each of first and second ports 20, 22 disposed in the open orientation concurrent to the other of said first and second ports 20, 22 disposed in the closed orientation is described herein and represented in at least FIGS. 1, 3-5 of the valve assembly 10 and FIGS. 6-9 of the valve assembly 10′.

With further regard to the first and second port 20, 22, each may include an external attachment segment 20″ and 22″ extending outwardly from the housing 12. Each such attachment segment 20″ and 22″ may be dimensioned and configured to establish appropriate connection and attachment with different sources/devices which facilitate introduction of fluid or instrumentation into the interior of the medical tubing 100 via passage through the hollow interior 14 of the housing 12 or 12′ and the primary connector 16. Accordingly, in more specific terms the first port 20 is structured to define a fluid flow port into the interior 14 of the housing 12 or 12′ and in communication with the medical tubing via the primary connector 16. In cooperation therewith, the second port 22 may be structured to define an intervention port for the introduction of instrumentation and/or other medical devices into the interior of the medical tubing via the hollow interior 14 of the housing 12 or 12′ and the primary connector 16. Therefore, one of the first and second ports 20, 22, such as second port 22, may be disposed and structured to facilitate the application of an interventional procedure. Such interventional procedure may include the introduction of instrumentation or medical devices such as, but not limited to, a suction catheter assembly, used to facilitate cleaning, maintenance, etc. of the interior of the medical tubing 100 concurrent to its application to the patient. The other of the first and second ports 20, 22, such as first port 20, is operative as a fluid flow port and may be connected to a vacuum or source of negative pressure, schematically represented as 200 in FIG. 1, thereby facilitating removal of any substances from the interior of the medical tubing, concurrent to it being applied to the patient. It is further recognized that either the first and second port 20, 22 may be externally closed or otherwise disposed out of direct communication with the hollow interior 14 during performance of a respiratory procedure and/or to maintain a sufficient positive and expiratory pressure (PEEP) level.

Additional structural and operative features of the valve assembly 10, include one or more embodiments thereof including a cover assembly attached to or integrated in the structure of the housing 12. As such, the cover assembly including cover members 36 and 38, are disposed such that the opposite longitudinal ends of the housing 12 are covered or enclosed and thereby segregate the hollow interior 14 of the housing 12 from an exterior thereof. In more specific terms as represented in FIGS. 1-5 the cover assembly may include cover members 36 and 38 disposed in covering relation to opposite longitudinal ends of the hollow interior 14 of the housing 12.

However, one or more additional embodiments of the present invention include the valve assembly 10′ as represented in FIGS. 6-9 and 11 comprising the housing 12′ having a T-shape configuration. Moreover, the T-shape housing 12′ includes at least one, but more practically a plurality of at least two supplementary ports 40 and 42 each disposed in fluid communication with the interior 14 of the housing 12′ and with the interior of the attached medical or endotracheal tubing via the primary connector 16. Further, each of the preferred two supplementary ports 40 and 42 are disposed at opposite longitudinal ends of the housing 12′ and may be disposed, structured and configured for operative connection with a ventilator/breathing apparatus facilitating the inflow and outflow of fluid, as the patient is breathing. Therefore, the T-shape embodiment of the adjustable valve assembly' facilitates maintenance thereof in operative attachment to the patient while selective disposition of the first and second ports 20, 22 in communication with the medical tubing via the hollow interior 14 of the housing 12′ and the primary connector 16.

Therefore, the structural and operative features of the adjustable valve assembly 10 and 10′ of the present invention eliminate the need to disassemble part of the respiratory support system for purposes of adding or connecting equipment intended maintain the medical or endotracheal tubing and its operation. Such intervention, as indicated above may include, but not be limited to, a suction catheter into the artificial airway. Fluid and like contaminants can then be suctioned from the patient without disconnection of the respiratory support system and eliminate dangerous conditions. It has been commonly recognized that the interruption of respiratory support during such an interventional procedure may result in a patient's blood oxygen often dropping and carbon monoxide possibly reaching unacceptable levels. Further, unless a sufficient positive and expiratory pressure (PEEP) level is maintained there is a possibility that the lungs of the patient will collapse. Moreover, procedures of this type may have to be performed numerous times per day and during relatively long periods of hospitalization of the patient.

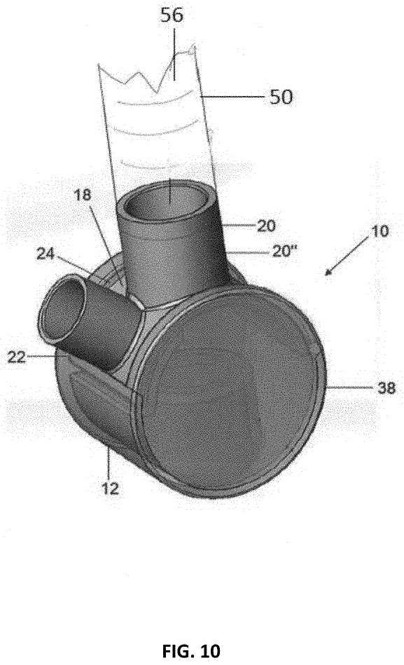

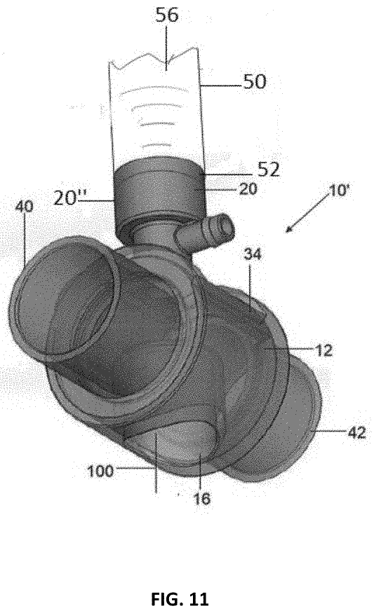

Yet additional features of the present invention are schematically represented in the embodiments of FIGS. 10 and 11. These structural features facilitate the sterility of any instrumentation such as, but not limited to, a suction catheter which would be disposed into the interior of the valve 10 or 10′ through either of the first or second ports 20 or 22 as the instrumentation passes into the interior of an endotracheal tube or other medical tubing disposed in the bronchial area or other portion of the patient's body.

More specifically, a flexible, collapsible material sleeve 50 may include one end 52 secured to at least one of the first or second ports 20 or 22, such as at about the attachment segments 20″ and 22″ the other end of the sleeve 50 would be secured to a portion of a suction catheter or other instrumentation at a location which would facilitate the enclosure of the sleeve 50 about a portion of the catheter or other instrumentation being inserted through the valve assembly 10 or 10′ into the interior of the medical tubing within the patient.

Therefore, a preferred fixed connections of opposite ends of the sleeve 40 respectively to the suction catheter assembly and to the adjustable valve assembly 10 or 10′, as well as the flexible material from which the sleeve 50 is formed, facilitates the inward collapse, compacting and/or folding of the sleeve upon itself and along its length, concurrently to the intended instrumentation being introduced through the first or second port 20 and 22 of the adjustable valve assembly 10 and into the interior of the endotracheal tube or other medical tubing within the patient. As indicated above, the inherent flexibility of the material from which the elongated sleeve is formed facilitates its lengthwise collapse or folding of the sleeve 50 upon itself at the same time the instrumentation is passing into and through the adjustable valve assembly 10 or 10′. After the suction catheter assembly or other instrumentation has performed the intended drainage/cleaning procedure of the interior of the endotracheal tube or other medical tubing within the patient, the withdrawing of the catheter assembly from the medical tubing, through the valve assembly 10 or 10′, will result in the concurrent expansion and/or elongation of the sleeve 50 into its original elongated configuration. Such elongation of the sleeve 50 will dispose it in the original overlying, enclosing relation to the inserted portion of the catheter assembly upon the respective withdrawal thereof from the medical tubing and the adjustable valve assembly 10 or 10′. As a result, the sleeve 50 will then be disposed in overlying covering and somewhat isolating relation to the inserted portion of the catheter assembly, thereby allowing it to be reused, while maintaining the sterility thereof, except for exposure to the body lumen or medical tubing.

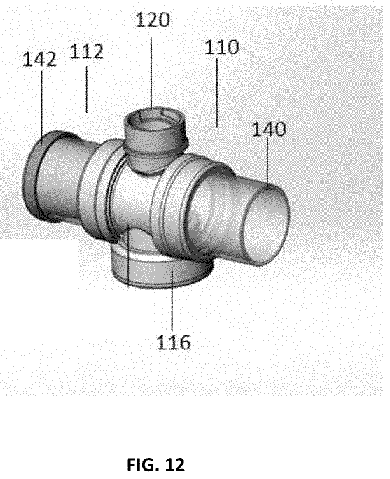

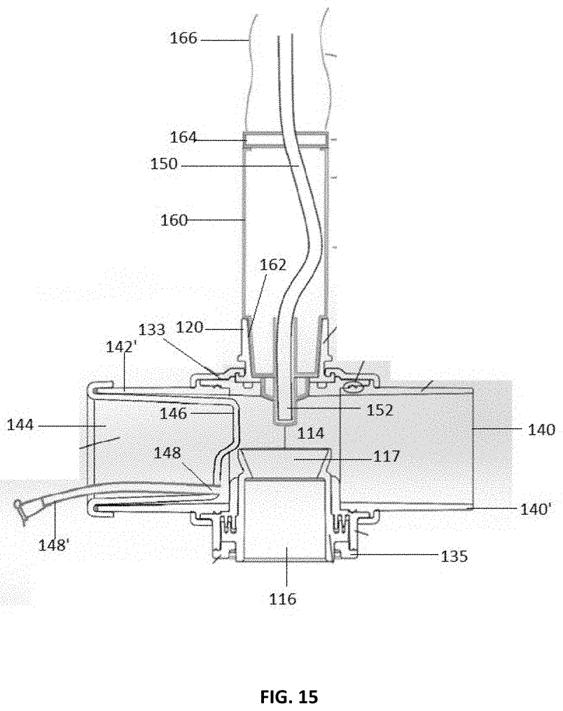

Yet another preferred embodiment of the valve assembly is represented in FIGS. 12-15 and is generally indicated as 110. In more specific terms, the valve assembly 110 includes a housing generally indicated as 112 including an introduction port 120, having connecting sleeve 120′. As discussed in greater detail hereinafter, with reference to FIG. 15, the introduction port 120 is structured to facilitate the interconnection of a catheter 150, which may be a steerable catheter, or like medical device to the housing 112 in a manner which facilitates its passage into the hollow directly into the interior of the airway port 116 and artificial airway connected thereto.

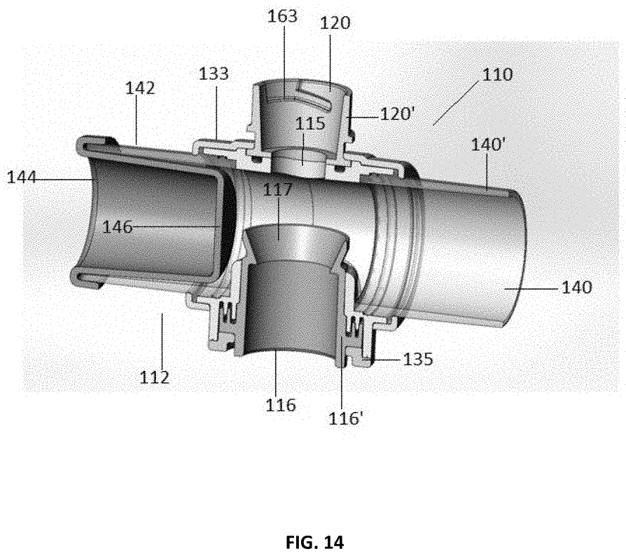

In addition, the housing 112 of the valve assembly 110 includes a primary or airway port 116, including connecting sleeve 116′ which is disposed in structured to facilitate attachment of the housing 112 to an artificial airway. The housing 112 also includes a plurality of supplemental ports 140 and 142 respectively including connecting sleeve 140′ in 142′ . Both the supplemental ports 140 and 142 are disposed in communicating relation to the hollow interior 114 of the housing 112. Structural and operative details of the interconnection of the introduction port 120 airway port 116 and supplemental ports 140 and 142 include retainer members 133 and 135 respectively disposed to retain the airway port 116 and the introduction port 120 and their respective connecting sleeves 116′ in 120′ in their respective operative orientations as represented at least in FIGS. 14 and 15. The supplemental ports 140 and 142 and their respective connecting sleeve 140′ in 142′ are similarly retained in the operative position by virtue of their at least partial enclosure or connecting interaction with the retainer 133 as represented in FIG. 14. It is of further note that cooperative structuring of the retainers 133 and 135 allow rotational or “swivel” type movement of the airway port 116 and/or connecting sleeve 116′ as well as at least the supplemental ports 140 and 142 and/or connecting sleeves 140′ in 142′.

Yet additional structural and operative features of the valve assembly 110 comprise the inclusion of a guide structure within the hollow interior 114 of the housing 112. As should be apparent hereinafter, such guide structure will assure that a steerable catheter 150, as schematically represented in FIG. 15, which may include a deflected angle, offset tip 152, will easily and efficiently pass into the interior 114 of the housing 112 through and from the introduction port 120 without getting caught or interfered with within the hollow interior 114. As such, the catheter 150 and catheter tip 152 will pass from the interior of the introduction port 120, through the hollow interior 114 and directly into the interior of the airway port 116 and thereafter to the artificial airway connected thereto, wherein the artificial airway is not shown for purposes of clarity.

In more specific terms, at least one additional embodiment of the valve assembly 110 includes the aforementioned guide structure including an expanded inner open end 117 of the airway port 116. Such expanded configuration may be at least partially defined and further described as an outwardly flared or funnel configuration, as clearly represented in FIGS. 14 and 15. Further, the outward flaring or expanded orientation of the inner open and 117 and its disposition on the interior 114 of the housing 112 relative to the interior open end of the introduction port 120 and/or access port 115 will facilitate the catheter tip 152 passing into the interior of the airway port 116 by virtue of the funnel like configuration of the inner open end 117. As described in greater detail with primary reference to FIG. 15 the expanded configuration of the inner open end 117 also serves to prevent or at least restrict the passage of collected secretions within the hollow interior 114 from passing into the airways port 116.

In at least one additional embodiment of the valve assembly 110, the guide structure further comprises the inclusion of a seal 144 removably disposed within the interior of at least one of the supplementary ports 140 or 142. As represented in FIGS. 14 and 15, the seal 144 is dimensioned and configured to block or prevent entrance of any instrumentation into the interior of the supplemental port 142 by virtue of an at least partially sealed engagement with the interior surface of the supplemental port 142. Further, the seal 144 includes an inner closed-end 146, which is disposed in directly adjacent relation to the inner ends 117 of the airway portal 116 and introduction port 120. As such, the closed-end 146 of the seal 144 is disposed and configured to effectively deflect the tip 152 of the catheter 150, once disposed on the interior 114 of the housing 112 into inserting relation to the expanded open end 117 of the airway port 116. As noted, the guide structure including the expanded open end 117 and the seal structure 144 are disposed in structured to maintain the catheter tip 152, which is deflected or angled outwardly from a coaxial center from the remainder of the catheter 150, from passing inadvertently into the interior of either of the supplemental ports 140 and 142. Further, the closed end 146 of the seal 142 is provided in cooperation with the open expanded and 117 of the airway port 116 to further assure accurate passage of the tip 152 and the remainder of the catheter 150 from and through the introduction port 120 into the hollow interior 114 and there from into and through the expanded open end 117 and through the interior of an airway port 116 into the artificial airway connected thereto.

As also represented in FIG. 15, additional structural operative features of the valve assembly 110 is the inclusion of a secretions discharge port generally indicated as 148 the discharge port 148 is located adjacent to or integrated with the seal 144 in fluid communication with the area surrounding portions of the airway port 116 which are disposed within the hollow interior 114 as represented. This area surrounding the interior portions of the airway port 16 is an area where secretions may collect. Therefore, the discharge port 148 is disposed in fluid communication with this area and includes a discharge conduit as at 148′ serving to direct any secretions collected from this area and/or within the interior 114 to the exterior of the housing 112. In addition, the outwardly expanded configuration of the inner open end 117 extends sufficiently above or outward from the area surrounding the entrance to the airway port 116 thereby further preventing or significantly restricting the passage of collected secretions into the interior of the airway port 16.

Further with regard to FIG. 15, a coupler structured 160 may have its proximal end 162 cooperatively structured with the introduction port 120 so as to be interconnected thereto, such as by connecting segment 163 in a removably retained manner on the housing 112. In at least one embodiment, the connecting segment 163 may be, but is not limited to, a bayonet type connecting structure. The interior of the coupler structure 160 facilitates the insertion of the aforementioned steerable catheter 150 and the distal end 162 may be further structured to engage, at least initially, the catheter tip 152 to facilitate its direct introduction into the interior 114 and therefrom into the interior of the airway port 116. The coupler's structure 160 may include a sealing cap at 164 which may be disposed in interconnecting relation between a sterile sleeve 166 and the outer or distal end of the coupler structure 160. In addition, the structural configuration and disposition of the distal end 162 of the coupler structure 160 may further facilitate the guidance of the catheter tip 152 into the hollow interior 114 and thereafter into the airway port 116 through the expanded open end 117. As such the distal end 162 may be considered part of the aforementioned guide structure.

With further regard to the embodiment of FIG. 15, yet additional structural features of the coupler 160 and more specifically the distal and 162 could be the inclusion of an “apertured construction” formed in the tubular portion of the distal end 162 that is represented in retaining or surrounding relation to the tip 152. Such an apertured construction may include one or more perforations, slits, etc. formed in the tubular portion of the distal end 162 which would allow airflow therethrough. Such apertured construction would make the presence of this tubular portion less obtrusive to airflow passing through the hollow interior 114. In turn this will allow the tubular portion to extend much farther into the hollow interior 14 closer to the entrance to the airway port 116. In turn this would improve the control of the catheter tip 150 while facilitating airflow across and/or through the hollow interior 114 between the one or more supplemental ports and the airway port 116. In addition, the same “apertured construction” may be included, at least in part into the expanded or final-shaped entrance 117 in a manner which would not negate the advantage of keeping secretions dripping down from within the hollow interior 114 into the entrance of the airway port 116.

Yet additional structural modifications in one embodiment of the present invention could include the coupler 160 having an interior telescopic sleeve which retracts or is movable from an interior position within the coupler 162 an extended position through the coupler 160 into the hollow interior 114 to an area closer to the entrance 117 of the airway port 116. Such an extended telescopic inner sleeve will allow the adjustment thereof to further facilitate guidance of the catheter tip 150 into the airway port 116. Once the catheter tip 152 and remainder of the catheter is withdrawn, the inner sleeve is also withdrawn into the interior of the coupler and possibly part way into the interior of the sterile sheath or sleeve 166. The advantage of such an additional structural modification would be a maximum or optimized control of the introduction of the catheter tip into the airway port 116.

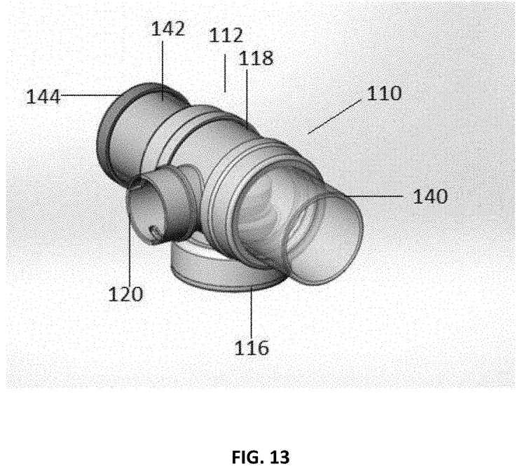

Yet additional structural operative features of the valve assembly 110 include the introduction port 120 being mounted on and movable with a closure segment 118, relative to the housing 112. Further, upon a rotational or swivel type movement of the closure segment 118, the introduction port 120 will be disposable between an open position as represented in FIG. 12 and a closed position as represented in FIG. 13. When in the open position of FIG. 12 the introduction port 120 is disposed in substantially linearly and/or coaxial aligned relation with the interior of the airway port 116, as represented in FIGS. 14 and 15. To the contrary, when in the closed orientation, the introduction port 120 is movable with the closure segment 118 relative to the housing 112 into non-aligned relation to the interior of the airway port 116. Therefore, the valve assembly 110 to the present invention further includes the closure segment 118 operative substantially similar to the closure segment 18 in the embodiments of FIGS. 1-11, as set forth above. More specifically, the introduction port 120 is mounted on and movable with the closure segment 118, to facilitate its selective positioning between the open orientation of FIG. 12 and the closed orientation of FIG. 13. When in the closed orientation, the dimension and configuration of the closure segment 118 allows it to be disposed in overlying, covering and closing relation to the access opening 115 of the housing 112, leading directly to the hollow interior 114. As set forth above, when the introduction catheter 120 is in the open orientation the interior of the introduction port 120 is in directly linearly aligned and/or coaxial relation to the access opening 115 as well as that of the airway port 116.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A valve assembly structured for regulating communication between medical tubing, said valve assembly comprising:
a housing including a hollow interior,
an airway report disposed on said housing and structured for attachment to an artificial airway,
an introduction port connected to said housing in communicating relation with said hollow interior; said introduction port disposed and structured to interconnect a catheter to said hollow interior of said housing, and
a guide structure disposed within said housing; said guide structured disposed and configured to direct the catheter from said introduction port, through said hollow interior into said airway port.

2. The valves assembly recited in claim 1 wherein said guide structure comprises an expanded open inner end of said airway port.

3. The valve structure as recited in claim 2 wherein said expanded open inner end comprises an outwardly flared configuration.

4. The valve structure as recited in claim 3 wherein said expanded open inner end comprises a substantially funnel-shaped configuration.

5. The valve structure as recited in claim 2 wherein said expanded open inner end comprises a substantially funnel-shaped configuration.

6. The valve assembly as recited in claim 2 further comprising a plurality of supplementary ports each connected to said housing in communicating relation with said hollow interior; said guide structure further comprising a seal structure removably disposed within at least one of said supplementary ports.

7. The valve assembly as recited in claim 6 wherein said seal structure includes a closed end disposed and configured to deflect the catheter from said introduction port through said hollow interior into said airway port.

8. The valve assembly as recited in claim 1 further comprising a plurality of supplementary ports each connected to said housing in communicating relation with said hollow interior; said guide structure comprising a seal structure removably disposed within at least one of said supplementary ports.

9. The valve assembly as recited in claim 8 wherein said seal structure includes an inner closed end disposed in adjacent relation to inner ends of said introduction port and said airway port.

10. The valve assembly as recited in claim 9 wherein said closed end is disposed and configured to deflect the catheter from said introduction port through said hollow interior into said airway port.

11. The valve assembly as recited in claim 8 further comprising a secretions removal port disposed adjacent said seal structure and structured to remove secretions from said hollow interior to an exterior of said housing.

12. The valve assembly as recited in claim 8 wherein each of said supplementary ports includes a connecting sleeve rotationally connected to said housing.

13. The valve assembly as recited in claim 8 wherein said airway port includes a connecting sleeve movably connected to said housing.

14. The valve assembly as recited in claim 1 further comprising a closure segment movably connected to said housing, said introduction port connected to said closure segment and movable therewith between open and closed orientations; said open orientation comprising said introduction port disposed in substantially coaxial relation to said airway port.

15. The valve assembly as recited in claim 13 further comprising an access opening formed in said housing in communicating relation with said hollow interior, said closed orientation of said introduction port comprising said closure segment movable on said housing in overlying relation to said access opening, concurrent to said introduction port disposed in no-aligned relation to said airway port.

16. The valve assembly as recited in claim 1 further comprising a coupler structure dimension to removably retain a catheter on the interior thereof; said coupler structure includes one end cooperatively structured with said introduction port to secure said coupler structure and a catheter contained therein in communicating relation with said hollow interior of said housing.

17. A valve assembly structured for regulating communication between medical tubing, said valve assembly comprising:
a housing including a hollow interior,
an airway report disposed on said housing and structured for attachment to an artificial airway,
a plurality of supplementary ports each connected to said housing in communicating relation with said hollow interior,
an introduction port connected to said housing in communicating relation with said hollow interior; said introduction port disposed and structured to interconnect a catheter to said hollow interior of said housing,
a guide structure disposed within said housing; said guide structured disposed and configured to direct the catheter from said introduction port, through said hollow interior into said airway port, and
guide structure comprising an expanded open inner end of said airway port having a substantially funnel shaped configuration disposed in guiding relation to a catheter tip passing through said hollow interior.

18. The valve assembly as recited in claim 17 wherein said guide structure further comprises a seal structure removably disposed within at least one of said supplementary ports; said seal structure including a closed end disposed and configured to deflect the catheter from said introduction port through said hollow interior into said airway port.

19. The valve assembly as recited in claim 18 further comprising a secretions removal port disposed within said hollow interior adjacent said seal structure; said secretions port structured to remove secretions from said hollow interior to an exterior of said housing.

20. The valve assembly as recited in claim 17 wherein each of said plurality of supplementary ports and said airway port includes a connecting sleeve rotationally connected to said housing.
